(19) United States
(12) Patent Application Publication
(10) Pub. No.: US 2025/0354968 A1
(43) Pub. Date: Nov. 20, 2025

(54) MULTI-PRESSURE SAMPLING FOR IMPROVING THE PERFORMANCE OF ELECTRONIC NOSES USING GAS ADSORBING SENSING ELEMENTS

(71) Applicant: UNIVERSITY OF PITTSBURGH-OF THE COMMONWEALTH SYSTEM OF HIGHER EDUCATION, PITTSBURGH, PA (US)

(72) Inventors: BRIAN A. DAY, PITTSBURGH, PA (US); CHRISTOPER ELI WILMER, PITTSBURGH, PA (US)

(73) Assignee: UNIVERSITY OF PITTSBURGH-OF THE COMMONWEALTH SYSTEM OF HIGHER EDUCATION, PITTSBURGH, PA (US)

(21) Appl. No.: 18/872,470

(22) PCT Filed: Jul. 24, 2023
(86) PCT No.: PCT/US2023/070839

§ 371 Date: Dec. 6, 2024

Related U.S. Application Data
(60) Provisional application No. 63/369,319, filed on Jul. 25, 2022.

(51) Int. Cl. G01N 33/00; G01N 29/02; G01N 29/22

(52) U.S. Cl. CPC G01N 33/0016; G01N 29/022; G01N 29/222; G01N 33/0031; G01N 2291/021

(57) ABSTRACT

A gas sensor array system includes a gas sensing chamber, a sensor array provided within the gas sensing chamber and including a plurality of gas sensing components each gas comprising a gas adsorbing material (such as an MOF material), and wherein the gas adsorbing material is different for each of the plurality of gas sensing components. The system also includes a gas source for supplying a gas sample to be measured, a gas pressure regulator structured and configured for selectively adjusting a pressure of the gas mixture suppled to the gas sensing chamber, and a controller structured and configured to (i) control the gas pressure regulator to selectively adjust the pressure of the gas sample to a number of pressure levels, and (ii) while the gas sample is provided at each pressure level, make a gas parameter measurement using outputs of one or more of the gas sensing components.

### STATEMENT OF GOVERNMENT INTEREST

This invention was made with government support under grant #1937179 awarded by the National Science Foundation (NSF). The government has certain rights in the invention.

### FIELD OF THE INVENTION

The disclosed concept relates generally to gas sensing systems, and, in particular, to gas sensor arrays, also referred to as electronic noses, that utilize gas adsorbing gas sensing elements, such as, without limitation, metal-organic frameworks (MOFs), as the sensing materials wherein multi-pressure sampling is used for improving the performance of the electronic noses.

### BACKGROUND OF THE INVENTION

Over the past few years, there has been a renewed interest in gas sensing arrays, known as electronic noses, due to improvements in sensing materials and advances in computational analysis techniques. In particular, research in the field of electronic noses has benefited from the explosive growth of research into metal-organic frameworks (MOFs) over the past two decades. MOFs are a large class of chemically and structurally diverse nanoporous crystalline materials with very high internal surface areas. They exhibit impressive and diverse gas adsorption properties, an important feature for the sensing elements of electronic noses, where high signal-to-noise ratios and cross-sensitivity are needed. To date, many different MOFs have been synthesized, and over 500,000 have been predicted.

Recently, research in MOF-based gas sensing has become less focused on materials engineering and more focused on materials selection and discovery. A common current approach is to select a synthesizable MOF (or set of MOFs) with an appropriate baseline pore size and chemistry and further engineer the pores via functionalization for improved sensing. These approaches are accelerated by computational techniques for modelling gas adsorption, such as grand-canonical Monte Carlo (GCMC) simulations, which makes the initial selection of materials much easier.

Compared with gas-chromatography mass-spectrometry (GCMS), electronic noses tend to be low-cost, highly portable devices, with fast response times, but at the expense of sensitivity and selectivity. Even so, these features open many exciting application areas, including industrial process monitoring, environmental monitoring, security, and of particularly high interest, disease diagnostics. The challenge for electronic noses becomes improving the performance without sacrificing other desirable features, such as speed and portability.

The performance of sensor arrays can be improved, in principle, by the addition of more complementary sensing elements. For this reason, much of the work in the field of electronic noses to date has focused on selecting the best materials for many-element sensor arrays and examining how the performance of the devices improves with array size. More specifically, such electronic noses typically tend to be comprised of MOF-films deposited onto mass-responsive sensors, such as quartz-crystal microbalances (QCM) or surface acoustic wave (SAW) devices, with the change in mass due to adsorption being measurable. With a working knowledge of how the adsorbed mass changes as a function of the bulk gas composition for each MOF in the array, the composition of an unknown gas mixture can be determined from only the detected mass values (that is, assuming the array has the needed signal-to-noise ratios and cross-sensitivities).

In previous work, a set of 50 MOFs were examined for methane-in-air sensing, carbon dioxide-in-air sensing, and even ammonia-in-breath sensing for kidney disease detection. However, as more complex gas mixtures (i.e., more components and lower concentrations of important gases) were explored, it became increasingly difficult to find MOFs with the adsorption behaviors needed to design a high-performing array, often due to the competitive adsorption of other gases in the sample. For example, when examining parts-per-million quantities of methane and hydrogen in breath, where the gases must compete with elevated quantities of carbon dioxide, simulations in the previous work of the present inventors predicted ultra-low adsorption of methane, and no adsorption of hydrogen, making the sensing problem impossible. Conversely, when examining strongly adsorbing gases, simulations showed that the MOFs often saturate at very low concentrations, rendering quantification of those gases impossible beyond a certain limit.

Finding MOFs with an appropriate change in mass as a function of composition for these gases, both strongly and weakly adsorbing, has proven difficult. Finding MOFs with appropriate cross-sensitivities for use in sensor arrays has also proven difficult.

### SUMMARY OF THE INVENTION

These needs, and others, are met by a gas sensor array system that includes a gas sensing chamber, and a sensor array provided within the gas sensing chamber, the sensor array including a plurality of gas sensing components, wherein each gas sensing component of the plurality of gas sensing components comprises gas adsorbing material, such as, without limitation, a metal-organic framework (MOF) material, and wherein the gas adsorbing material is different for each of the plurality of gas sensing components. The system also includes a gas source for supplying a gas sample to be measured, a gas pressure regulator coupled to the gas source and the gas sensing chamber, the gas pressure regulator being structured and configured for selectively adjusting a pressure of the gas mixture suppled to the gas sensing chamber, and a controller structured and configured to (i) control the gas pressure regulator to selectively adjust the pressure of the gas sample provided to the gas sensing chamber to a number of pressure levels, and (ii) while the gas sample is provided at each pressure level, make a gas parameter measurement on the gas sample using outputs of one or more of the gas sensing components.

In another embodiment, a gas sensing method is provided that includes providing a sensor array including a plurality of gas sensing components, wherein each gas sensing component of the plurality of gas sensing components comprises a gas adsorbing, such as, without limitation, an MOF material, and wherein the gas adsorbing material is different for each of the plurality of sensing components, providing a gas sample to the sensor array at number of pressure levels, and while the gas sample is provided at each pressure level, making a gas parameter measurement on the gas sample using outputs of one or more of the gas sensing components.

### BRIEF DESCRIPTION OF THE DRAWINGS

A full understanding of the invention can be gained from the following description of the preferred embodiments when read in conjunction with the accompanying drawings in which:

FIGS. 1 a-e show ternary plots of the adsorbed mass of hydrogen in MgMOF-74 as a function of composition according to an embodiment of the disclosed concept, and FIG. if shows the MOF of this embodiment;

FIGS. 2a-e show ternary plots of the adsorbed mass of methane in MgMOF-74 as a function of composition according to an embodiment of the disclosed concept, and FIG. 2f shows the MOF of this embodiment;

FIGS. 3a-e show ternary plots of the adsorbed mass of benzene in MOF-177 as a function of composition according to an embodiment of the disclosed concept, and FIG. 2f shows the MOF of this embodiment;

FIGS. 4a-e show ternary plots of the adsorbed mass of hydrogen sulfide in UiO-66 as a function of composition according to an embodiment of the disclosed concept, and FIG. 4f shows the MOF of this embodiment;

FIGS. 5a and 6a-c show the ternary probability plot and component probability plots of the best 3-element array at ambient pressure (1 bar) sensing conditions, respectively, and FIG. 5b and FIGS. 6d-f show the ternary probability plot and component probability plots of the best 3-element array using multiple pressures, respectively; and

FIG. 7 is a schematic diagram of a gas sensor array system (an electronic nose) 5 that implements the disclosed concept according to an non-limiting exemplary embodiment of the disclosed concept.

### DETAILED DESCRIPTION OF THE INVENTION

As used herein, the singular form of “a”, “an”, and “the” include plural references unless the context clearly dictates otherwise.

As used herein, the statement that two or more parts or components are “coupled” shall mean that the parts are joined or operate together either directly or indirectly, i.e., through one or more intermediate parts or components, so long as a link occurs.

As used herein, “directly coupled” means that two elements are directly in contact with each other.

As used herein, the term “number” shall mean one or an integer greater than one (i.e., a plurality).

As used herein, the term “controller” shall mean a programmable analog and/or digital device (including an associated memory part or portion) that can store, retrieve, execute and process data (e.g., software routines and/or information used by such routines), including, without limitation, a field programmable gate array (FPGA), a complex programmable logic device (CPLD), a programmable system on a chip (PSOC), an application specific integrated circuit (ASIC), a microprocessor, a microcontroller, a programmable logic controller, or any other suitable processing device or apparatus. The memory portion can be any one or more of a variety of types of internal and/or external storage media such as, without limitation, RAM, ROM, EPROM(s), EEPROM(s), FLASH, and the like that provide a storage register, i.e., a non-transitory machine readable medium, for data and program code storage such as in the fashion of an internal storage area of a computer, and can be volatile memory or nonvolatile memory.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

The disclosed concept will now be described, for purposes of explanation, in connection with numerous specific details in order to provide a thorough understanding of the subject invention. It will be evident, however, that the disclosed concept can be practiced without these specific details without departing from the spirit and scope of this innovation.

As described herein, the disclosed concept improves the performance of an gas adsorbing-based (e.g., MOF-based) sensor array by increasing the likelihood of finding the needed adsorption behaviors in the gas adsorbing-based (e.g., MOF-based) sensor array by modulating how the gas sensing components interact with gas mixtures. More specifically, the disclosed concept explores how the adsorption of gas mixtures into, for example, MOFs, changes as a function of system pressure, and the positive impact that multi-pressure sampling can have on the performance of gas sensing arrays. By designing and implementing arrays in which both multiple gas sensing components and multiple pressures are employed, the disclosed concept is able to modify adsorption behaviors such that it becomes easier to detect previously troublesome gases and increase the information content of the device without needing to synthesize more sensors, such as MOFs.

Moreover, the disclosed concept explores the use of variable system pressure as a means of improving sensitivity, selectivity, and increasing the information provided by each array. In connection with the disclosed concept, 9 different MOFS (HKUST-1, IRMOF-1, MgMOF-74, MOF-177, MOF-801, NU-100, NU-125, UiO-66, and ZIF-8) and 4 different gas mixtures, each containing nitrogen, oxygen, carbon dioxide, and exactly one of the either hydrogen, methane, hydrogen sulfide, or benzene, were explored. It was found that by lowering the pressure, oversaturated adsorption in MOFs can be prevented, and by raising the pressure, weakly adsorbing gases can be concentrated, in both cases improving detection with the resulting arrays. In some cases, changing system pressure yielded more information (as measured by the Kullback Liebler Divergence of the gas composition probability distributions) than including more MOFS. The disclosed concept thus demonstrates and quantifies how sensing at multiple pressures can increase information content and cross-sensitivity in MOF-based arrays, while limiting the number of unique materials needed in the device.

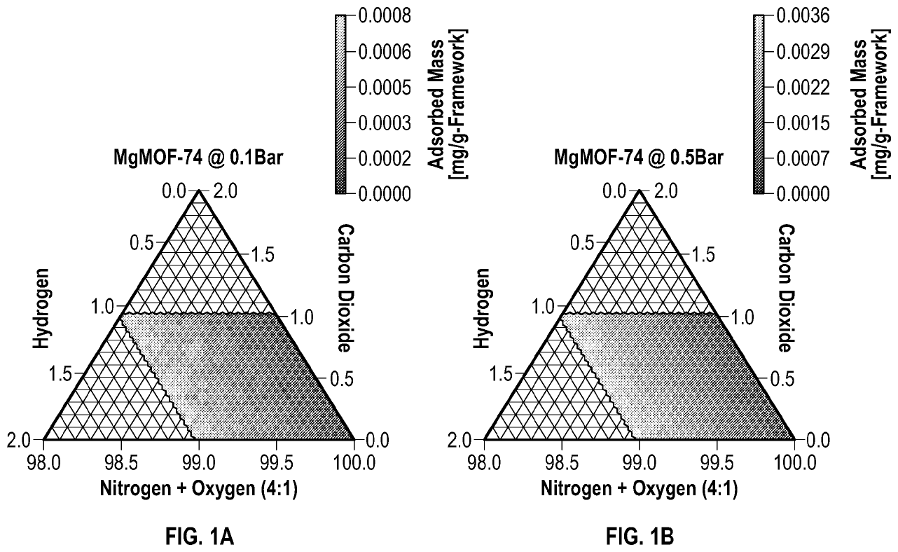

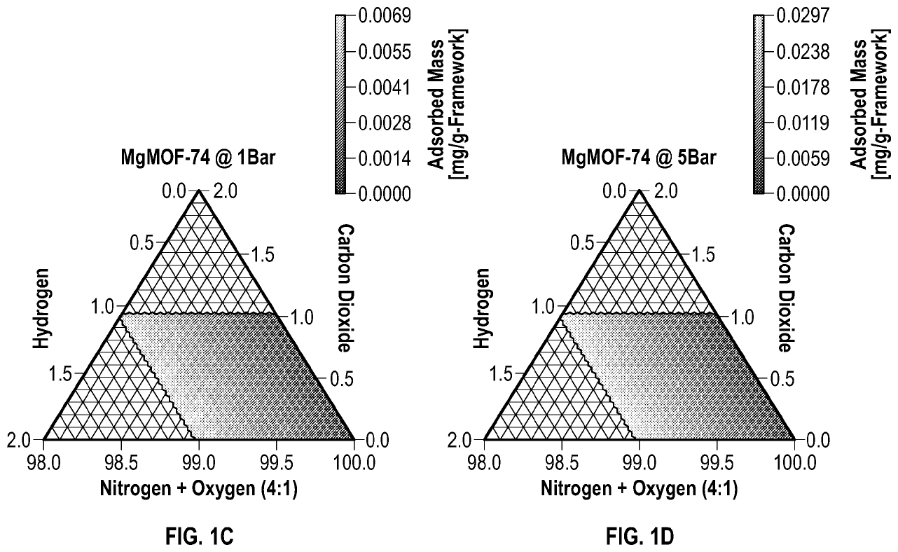

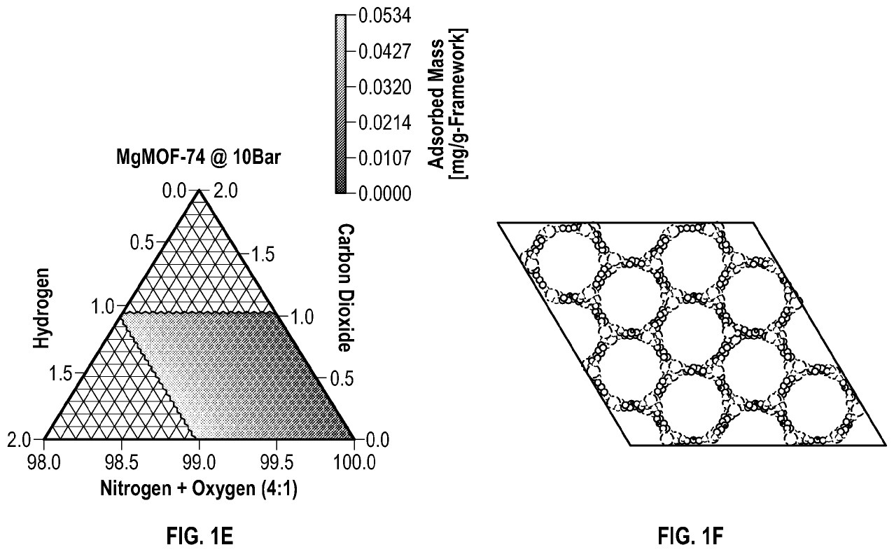

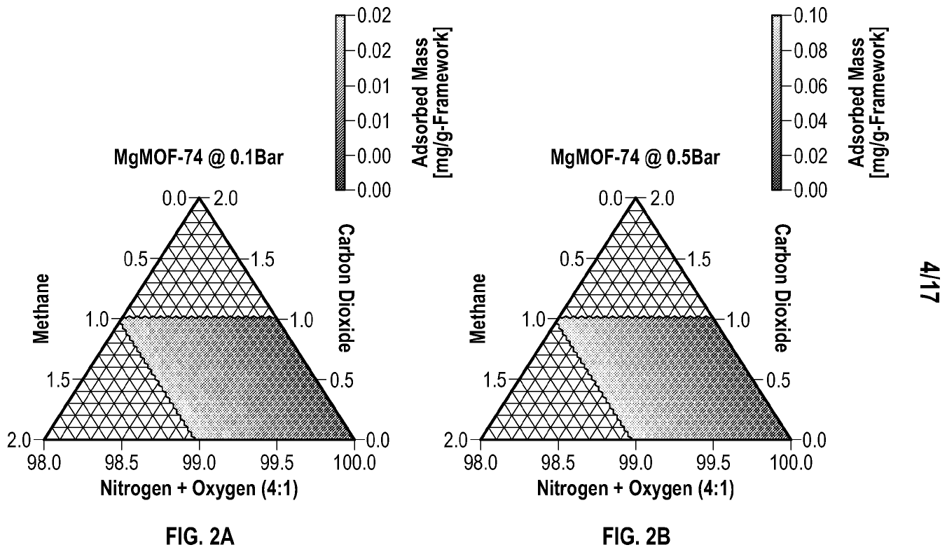

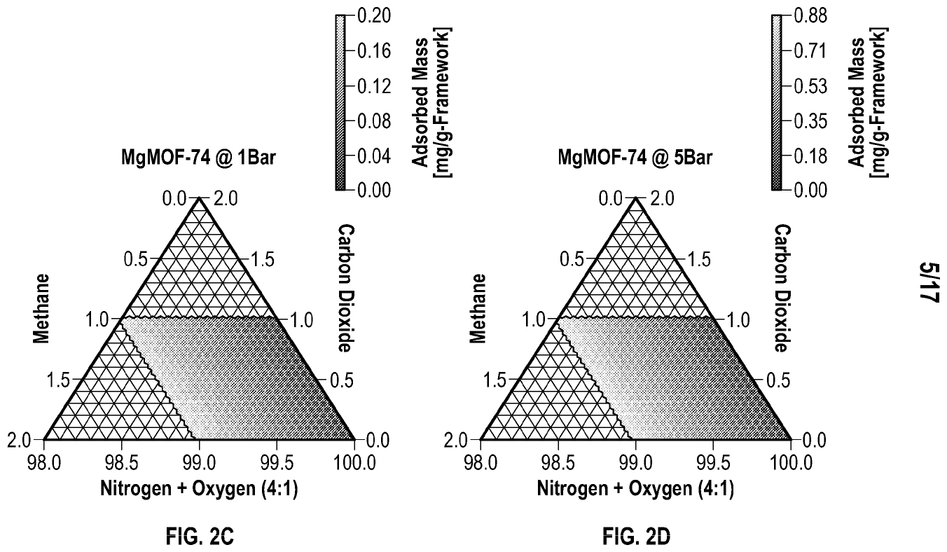

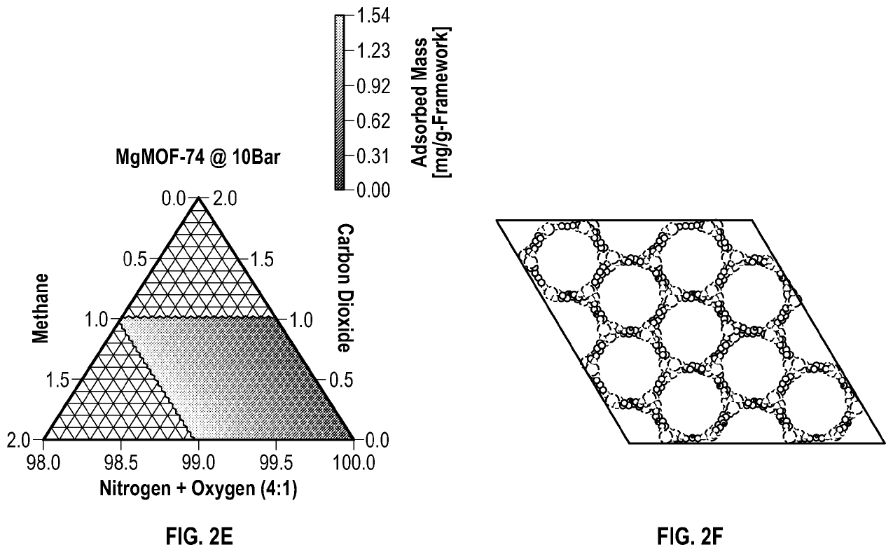

Methane and hydrogen are both small non-polar molecules and, as a result, are typically weakly adsorbing gases. Consequently, when exposing MOFs to complex gas mixtures, these gases will frequently make up only a small fraction of the total adsorbed mass, an effect which is exaggerated when these gases are present in very low concentration. In order to reliably detect and quantify gases with mass-based sensing, it generally helps to increase the amount in which they adsorb relative to other gas species. With this in mind, the present inventors hypothesized that by increasing the system pressure, the amount of gas adsorbed could be increased, particularly for small molecules, which should pack more efficiently than large molecules. As a result, the ability to detect gases like methane and hydrogen in complex gas mixtures can be improved. That said, the size difference between nitrogen (3.64 Å) and oxygen (3.46 Å) versus hydrogen (2.89 Å) and methane (3.80 Å) is not very significant, compared to a molecule like benzene (5.85 Å), so it is not apparent what impact pressure would have on the selectivity of adsorbed gases, especially in the presence of more strongly adsorbing small gases like CO₂. Fortunately, it is still possible that even if the selectivity does not change by increasing pressure, that increasing the total adsorbed mass could improve the performance of mass-based sensors simply by increasing the signal-to-noise ratio. One of the MOFs which best demonstrates the ability of high-pressure sensing to improve small molecule detection by concentrating them more strongly in the sensing material is MgMOF-74. FIGS. 1a to 1e show ternary plots of the adsorbed mass of hydrogen in MgMOF-74 as a function of composition and at the following pressures: a) 0.1 bar, b) 0.5 bar, c) 1 bar, d) 5 bar, and c) 10 bar. Note that the legend refers to the mass contribution of the hydrogen only, rather than the total adsorbed mass. FIG. if shows a 2×2×2 unit cell of the MOF projected down the c-axis. Thus, as seen in FIGS. 1a-1e, as the pressure of the system is increased, the total adsorbed mass of hydrogen increases sixty seven-fold from 0.1 bar (0.0008 mg/g-framework) to 10.0 bar (0.0534 mg/g-framework). Similarly, referring to FIGS. 2a-2f, the adsorption of methane undergoes an even larger a seventy seven-fold increase in mass from 0.1 bar (0.02 mg/g-framework) to 10.0 bar (1.54 mg/g-framework). Specifically, FIGS. 2a-2e show ternary plots of the adsorbed mass of methane in MgMOF-74 as a function of composition and at the following pressures: a) 0.1 bar, b) 0.5 bar, c) 1 bar, d) 5 bar, and e) 10 bar. Note that legend refers to the mass contribution of the methane only, rather than the total adsorbed mass. FIG. 2f shows a 2×2×2 unit cell of the MOF projected down the c-axis.

When the total adsorbed mass is too low, the mass detection limits of the device become significant and measurement noise overwhelms the signal. Hence, the significant increase in the adsorbed mass from 0.1 to 10 bar is beneficial. Additionally, at the higher pressures used, the Monte Carlo sampling underlying the simulation method employed converges more efficiently, decreasing the error especially for weakly adsorbing gases, as evidenced by the smoothness of the high-pressure plots.

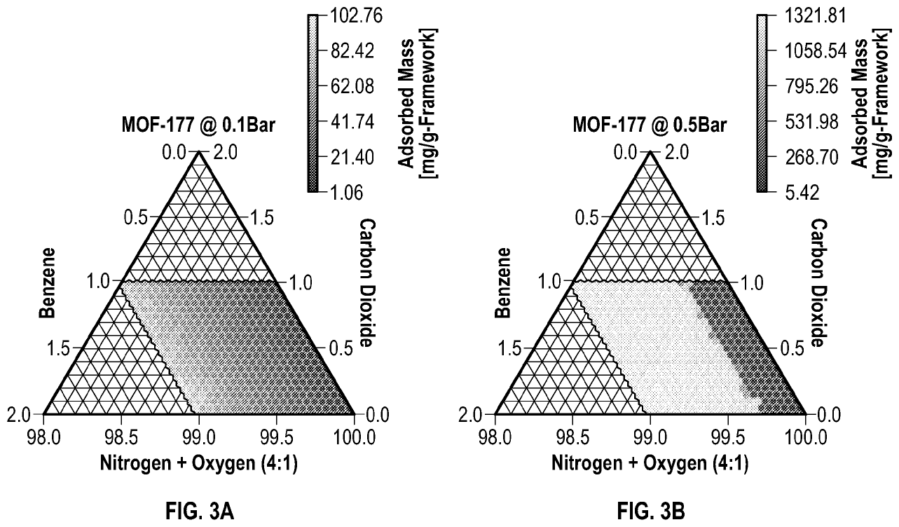

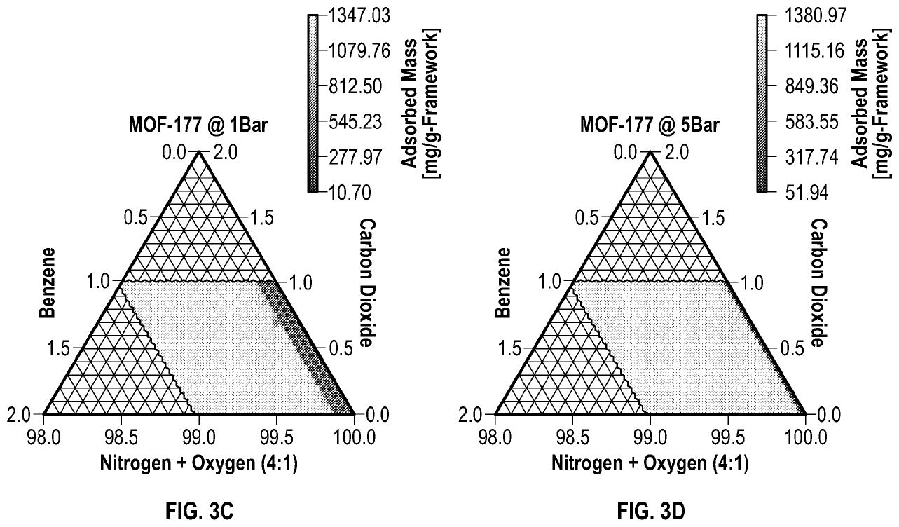

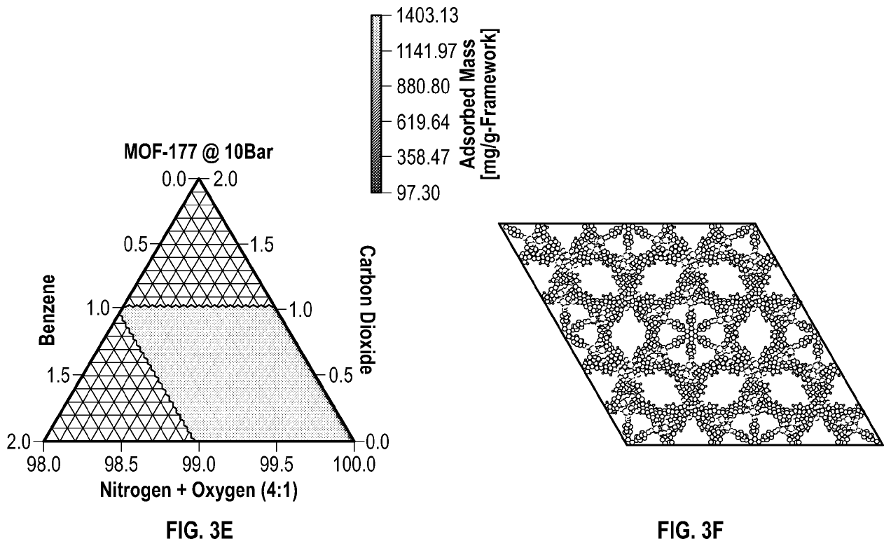

Although it is non-polar, benzene is generally a strongly adsorbing gas due to its large size. Even at low concentrations, it makes up a significant fraction of the total adsorbed mass in most MOFs and can rapidly saturate the sensor response (i.e., a change in the concentration does not result in a change in adsorbed mass). This not only makes benzene difficult to detect, but also the detection of non-benzene gases, since the MOFs lose sensitivity towards those gases in the presence of benzene. In order to improve the detection of benzene, the present inventors hypothesized that it would be beneficial to decrease the pressure, to the point that the sensor is no longer saturated, and changes in benzene concentration would again result in a change of mass. It was found that this effect does indeed occur and is demonstrated well by MOF-177. FIGS. 3a to 3e show ternary plots of the adsorbed mass of benzene in MOF-177 as a function of composition and at the following pressures: a) 0.1 bar, b) 0.5 bar, c) 1 bar, d) 5 bar, and e) 10 bar. Note that the legend indicates total adsorbed mass, rather than just the benzene contribution. FIG. 3f we show a 2×2×2 unit cell of the MOF projected down the c-axis. Note that the maximum observed total adsorbed mass at 0.1 bar is significantly lower than that observed at 0.5 bar and above. Even so, this decrease in total adsorbed mass is coupled with the necessary desaturation of the sensor, enabling a better ability to distinguish ambient benzene concentrations over this range. Improving benzene sensing by shifting to low pressures highlights an important concept of the sensing elements of electronic noses; the best elements are those in which the change in total adsorbed mass from one composition to another is greatest. It is easy to think that highly selective, highly adsorbing MOFs are best, and subsequently, that high mass loadings are universally desired. But as benzene demonstrates, this is not inherently true. At lower pressures, both the selectivity towards benzene and total adsorbed mass decreases, but sensing is still improved because the change in mass as a function of change in composition is improved. It should, however, be mentioned that for applications where benzene is present in extremely low concentrations (ppm and below), high pressures may not result in saturation of the sensor and may actually benefit from high pressures due to a concentrating effect similar to hydrogen and methane. In fact, one of the MOFs that was screened, NU-100, is more useful at high pressures when detecting benzene for this reason. Nevertheless, all other MOFs screened perform best at low pressures, and FIGS. 3a-3e demonstrate the potential benefits of low-pressure sensing.

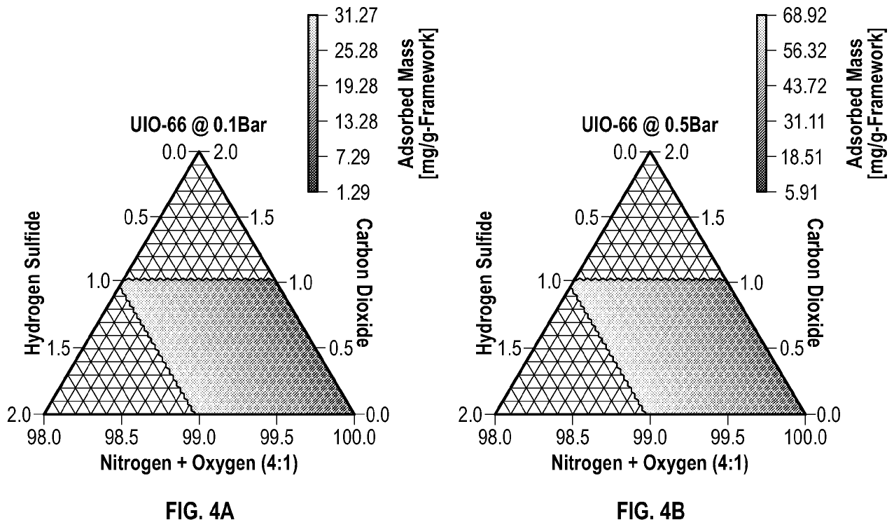

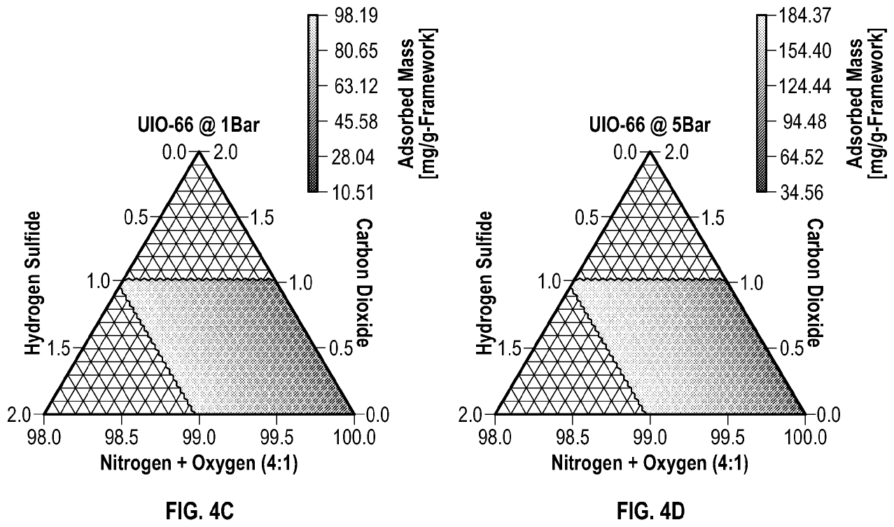

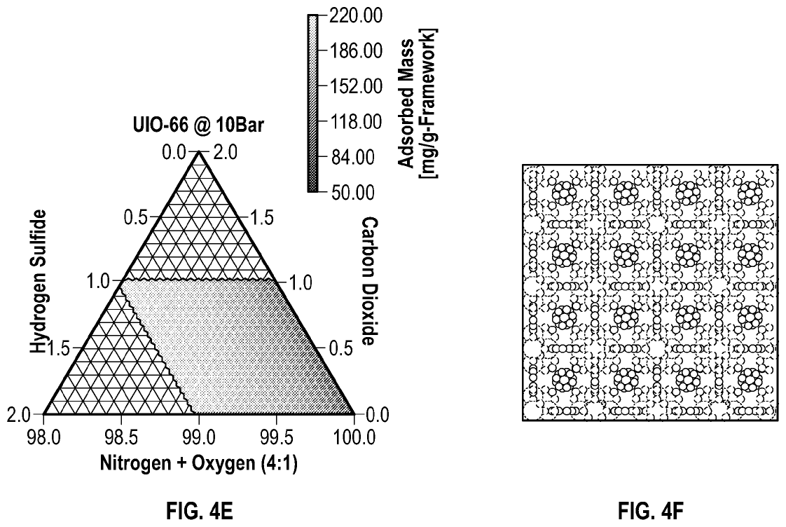

Hydrogen sulfide, like methane and hydrogen, is a small molecule, but it also has a strong dipole moment that typically leads to stronger adsorption within MOF pores. Although there are many MOFs which adsorb hydrogen sulfide appropriately for sensing at ambient pressure, the adsorption behavior can still be beneficially modified by changing system pressure. This is demonstrated well by UiO-66. FIGS. 4a-4e show ternary plots showing the adsorbed mass of hydrogen sulfide in UiO-66 as a function of composition and at the following pressures: a) 0.1 bar, b) 0.5 bar, c) 1 bar, d) 5 bar, and e) 10 bar. FIG. 4f shows a 2×2×2 unit cell of the MOF projected down the c-axis. At each of the simulated pressures, there is an appreciable change in the total adsorbed mass as a function of the composition (as opposed to a total adsorbed mass which is invariant of composition), meaning that each pressure is useful for determining the composition of the gas mixture. At first glance, at high pressures and high concentrations of hydrogen sulfide (as seen in FIGS. 4d and 4e), the mass response as a function of composition appears to flatten out (the change in adsorbed mass relative to the total adsorbed mass decreases). However, this apparent flattening of the response is sufficiently compensated by an overall increase in the total adsorbed mass such that absolute change in mass as a function of composition at high pressures is greater than that at lower pressures, thus offering better sensing performance. Nevertheless, it is very likely that there are other MOFs which would benefit more from low pressure sampling, when the increase in the total adsorbed mass at high pressures cannot compensate for this flattening out behavior.

For the 9 MOFs screened in connection with the disclosed concept, hydrogen sulfide sensing, like hydrogen and methane sensing, is easier at high pressures. But unlike hydrogen or methane, it is easy to envision an MOF for which the optimum sensing pressure is actually lower, especially if high concentrations are expected in the application, where saturation of the sensing material is more plausible.

Every possible array of each size and pressure was analyzed and its performance quantified with a KLD score. In general, array performance always improves with array size. The change in the performance of the best arrays as a function of array size, however, is minimal, suggesting that these arrays rely on only a few high-performing MOFs to make their predictions. It was found that pressure has a much more significant impact. For hydrogen, methane, and hydrogen sulfide containing gas mixtures, array performance improves specifically with higher pressure operation. In fact, of these three gases, hydrogen sulfide is the only one which exhibits a significant increase in performance beyond size 1 arrays. The jump in performance from size 1 to size 2 arrays for hydrogen sulfide, especially at high pressures, suggests that increasing pressure results in improvement to not just the individual adsorption behaviors, but also the cross-sensitivity of the elements. Even then, beyond size 2 arrays, the improvement in performance is again minimal, consistent with the idea that the best arrays rely on only a few elements. Conversely, the worst arrays generally improve with both pressure and array size. For example, the KLD scores of the worst arrays at 5 and 10 bar for hydrogen, methane, and hydrogen sulfide all increase steadily with array size. This is because more of the MOFs exhibit useful adsorption behavior at these pressures, even if they are noticeably outperformed by the best MOFs. However, at low pressures, the increase in performance as a function of array size is again limited. For example, with hydrogen sensing at 0.1 bar, the KLD score for the worst arrays is almost 0.0 until all 9 MOFs are used, suggesting that only one of the MOFs has any useful adsorption characteristics for detecting hydrogen. Similar behavior was observed for this and other gases at low pressures, suggesting that, under certain conditions, some the MOFs do contribute significantly to sensing performance.

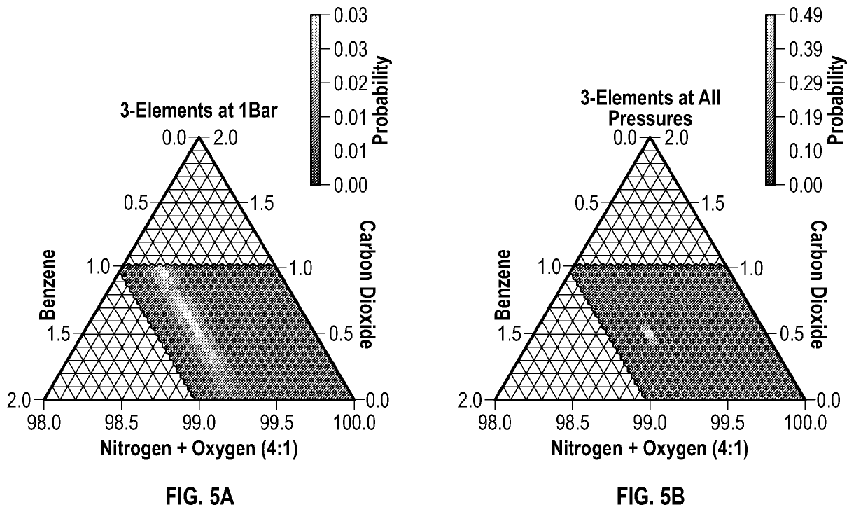

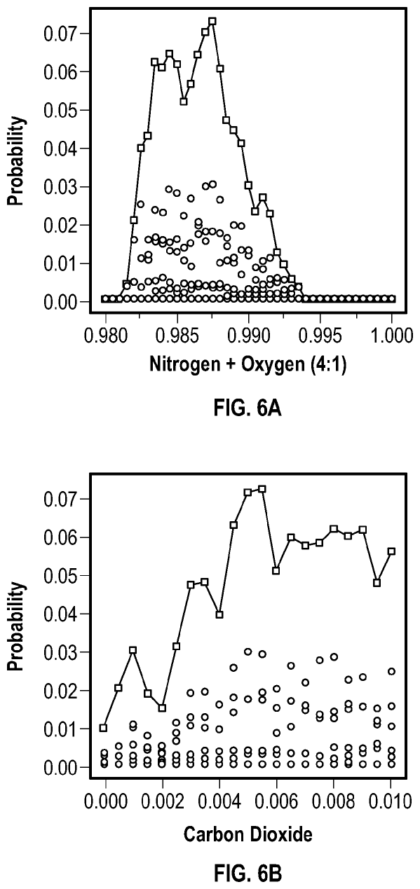

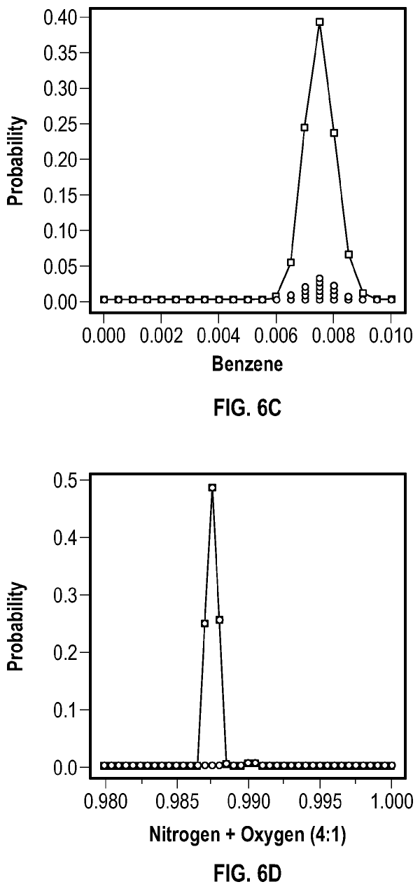

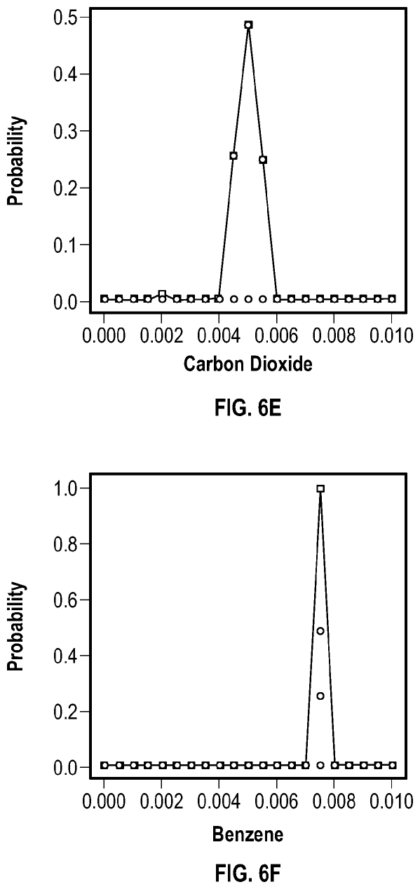

Together, these results highlight how varying pressure can change the approach to both the search problem (i.e., screening MOFs) and array design problem (i.e., choosing the correct combination of MOFs) central to the building an electronic nose. In terms of the search problem, for some gases it will be easier to find materials with useful adsorption behaviors by examining fewer MOFs at more pressures, rather than more MOFs at a single pressure. Similarly, in terms of array design, using small arrays at an optimized pressure or set of pressures is more beneficial than using large arrays at a single unoptimized pressure. On this note, since methane, hydrogen, and hydrogen sulfide benefit specifically from high pressures, there is only a marginal improvement in the performance of the all-pressure arrays when compared to the single-pressure arrays operating at high pressures. This does not mean, however, that there is never any benefit to operating at multiple pressures. With benzene sensing, most of the MOFs at atmospheric and high pressures saturate at very low concentrations, making detection of benzene beyond these concentrations practically impossible. By shifting to lower pressures, however, saturation occurs at higher benzene concentrations thus enabling detection. Given this, one might expect benzene to benefit specifically from low pressure sensing, just as hydrogen and methane benefited specifically from high pressures, but NU-100 exhibits unique behavior. It does not saturate at low benzene concentrations until operating at a pressure of 10 bar. At 5 bar, the change in mass as a function of benzene concentration is sharpest, making sensing at this pressure better than either low or atmospheric pressures. In fact, the only single element that outperforms NU-100 at 5 bar is IRMOF-1 at 0.1 bar 0.35 As a result, there is a noticeable improvement in the performance of all-pressure arrays, with all of the best all-pressure arrays of size 2 or more contain NU-100 and IRMOF-1. FIGS. 5a and 6a-c show the ternary probability plot and component probability plots of the best 3-element array at ambient pressure (1 bar) sensing conditions, respectively. FIG. 5b and FIGS. 6d-f show the ternary probability plot and component probability plots of the best 3-element array using multiple pressures, respectively. More specifically, FIGS. 5a-b show the probability vs. composition for a) the best 3-element array at 1 bar (NU-100, M 77, HKUST-1) and b) the best 3-element array at all pressures (NU-100, IRMOF-1, HKUST-1), and FIGS. 6a-f show the probability vs. component mole fraction for the best 3-element array at 1 bar (NU-100,

MOF-177, HKUST-1) for a) nitrogen/oxygen, b) carbon dioxide, and c) benzene and for the best 3-element array at all pressures (NU-100, MOF-177, HKUST-1) ford) nitrogen/oxygen, e) carbon dioxide, and f) benzene.

It is clear from these FIGS. 5 and 6 that, just by sampling a few additional pressures, the ability to detect gases can be dramatically improved. Although the 1 bar array does an acceptable job of detecting benzene, there is still a wide margin of error, and the prediction for air and carbon dioxide is very poor. With multiplex sensing, the prediction can be narrowed down to almost a single composition (i.e., all other compositions have a near-zero probability assigned to them). While it is interesting that for benzene, some MOFs performed best at both low and high pressures, and consequently there was a noticeable benefit for multiplex sensing, it is in general beneficial for sensing mixtures to consider multiple pressures. With most real gas mixtures being more complex, there will certainly be cases where detecting certain species of gases benefits from lower pressures while for others from high pressures, such as a system containing benzene and methane in air, which is relevant in natural gas processing

Thus, for all gas mixtures, the studied MOF arrays showed improved performance at non-atmospheric pressures. Furthermore, the information gain from sampling at multiple pressure sampling always improved performance relative to sampling at just atmospheric pressure. Detection of hydrogen, methane, and hydrogen sulfide benefited specifically from high pressures (greater than 1 bar), whereas benzene detection benefited mostly from low pressures (less than 1 bar). Exceptionally, the MOF NU-100 performed best for benzene sensing at 5 bar, and thus arrays for benzene sensing which contained NU-100 exhibited a notable improvement when sampling at multiple pressures. For most real gas mixtures, it is expected that sensing will benefit from leveraging both low and high pressures, as the gas mixtures will likely contain a combination of gases which are easier to detect at low pressures (e.g., benzene) and gases which are easier to detect at high pressures (e.g., hydrogen, methane, and hydrogen sulfide) In general, low-pressure operation seems to benefit the detection of strongly adsorbing gases which easily saturate sensors, and high-pressure operation is better for detecting dilute or weakly adsorbing gases. By exploring and operating at multiple pressures, it is easier to both find useful MOFs candidates and design cross-sensitive arrays. The disclosed concept therefore provides an improvement in the sensing capabilities of electronic noses while limiting the number materials, making device fabrication cheaper and easier.

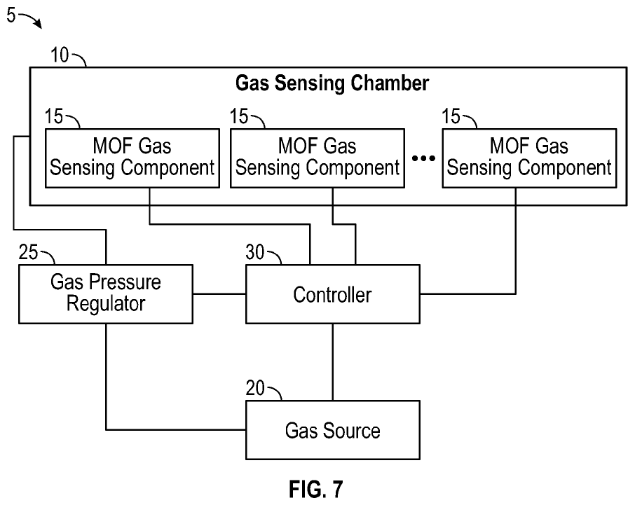

FIG. 7 is a schematic diagram of a gas sensor array system (an electronic nose) 5 that implements the disclosed concept according to an non-limiting exemplary embodiment of the disclosed concept. System 5 includes a gas sensing chamber 10. Gas sensing chamber 10 houses a gas sensing array comprising a plurality of MOF gas sensing components 15. Each MOF gas sensing component 15 includes an MOF film deposited onto a mass-responsive sensor device, such as a quartz-crystal microbalance (QCM) device or a surface acoustic wave (SAW) device that enables the change in mass due to gas adsorption to be measured. In the exemplary embodiment, the specific MOF of each MOF gas sensing component 15 is different, i.e., is made of a different MOF material structure. In the exemplary embodiment, the MOFs that are used to construct the array may be any of two or more of the following MOFs in combination: HKUST-1, IRMOF-1, MgMOF-74, MOF-177, MOF-801, NU-100, NU-125, UiO-66, and ZIF-8. In one particular exemplary embodiment, the array includes at least 9 MOF gas sensing components made of the following MOFs, respectively: HKUST-1, IRMOF-1, MgMOF-74, MOF-177, MOF-801, NU-100, NU-125, UiO-66, and ZIF-8. It will be appreciated that these disclosed embodiments are meant to be exemplary only, and that other types of MOFs in various combinations (with or without one or more of those just mentioned) may also be used within the scope of the disclosed concept.

System 5 also includes a gas source 20 that is structured to hold and selectively supply a gas mixture to be analyzed. Gas source 20 is coupled to a gas pressure regulator 25 structured and configured to selectively regulate the pressure of the gas that is provided to gas sensing chamber 10. Gas pressure regulator 25 is thus able to regulate the operating pressure of gas sensing chamber 10, and thus the operating pressure of system 5. System 5 further includes a controller 30 that is couped to gas sensing chamber 10, gas source 20 and gas pressure regulator 25 that is structured and configured for controlling the operation thereof. Specifically, controller 30 is able to control gas pressure regulator 25 in order to selectively control the pressure of the gas that is delivered and thus the operating pressure of system 5. In addition, controller 30 receives the output of each MOF gas sensing component 15. With knowledge of how the adsorbed mass changes as a function of the bulk gas composition for each gas sensing component 15 in the array, system 5 can determine the composition of an unknown gas mixture from only the detected mass values. In addition, gas pressure can be selectively regulated as described herein to optimize the performance of each MOF gas sensing component 15, and thus the overall performance of system 5. System 5 is thus able to use variable pressure to improve the sensitivity and selectivity of the array of MOF gas sensing components 15 and increase the information that is provided by the array of MOF gas sensing components 15.

While the disclosed concept has, for illustrative purposes, been described in connection with an electronic nose that employs an array of MOF-based gas sensing components, it will be appreciated that that is meant to be exemplary only. The disclosed concept may be used in connection with an array including other types of gas sensing components where gases adsorb onto the surface of the gas sensing component. Such alternative gas sensing components may include, for example and without limitation, sensors that use the following gas adsorbing materials: (1) thin film polymers, (2) activated carbon, (3) zeolites, (4) COFs (covalent organic frameworks), (5) aerogels, (6) metal-oxide surfaces, and (7) nanoporous/microporous materials.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of disclosed concept which is to be given the full breadth of the claims appended and any and all equivalents thereof.

What is claimed is:

1. A gas sensor array system, comprising:
a gas sensing chamber;
a sensor array provided within the gas sensing chamber, the sensor array including a plurality of gas sensing components, wherein each gas sensing component of the plurality of gas sensing components comprises a gas adsorbing material, and wherein the gas adsorbing material is different for each of the plurality of gas sensing components;
a gas source for supplying a gas sample;
a gas pressure regulator coupled to the gas source and the gas sensing chamber, the gas pressure regulator being structured and configured for selectively adjusting a pressure of the gas mixture suppled to the gas sensing chamber; and
a controller structured and configured to (i) control the gas pressure regulator to selectively adjust the pressure of the gas sample provided to the gas sensing chamber to a number of pressure levels, and (ii) while the gas sample is provided at each pressure level, make a gas parameter measurement on the gas sample using outputs of one or more of the gas sensing components.

2. The gas sensor array system according to claim 1, where each of the gas sensing components comprises a metal oxide framework (MOF) material as the gas adsorbing material, and wherein the MOF is different for each of the plurality of gas sensing components.

3. The gas sensor array system according to claim 2, wherein the plurality of gas sensing components comprises two or more gas sensing components, and wherein the MOF materials include two or more of HKUST-1, IRMOF-1, MgMOF-74, MOF-177, MOF-801, NU-100, NU-125, UiO-66, or ZIF-8.

4. The gas sensor array system according to claim 2, wherein the plurality of gas sensing components comprises nine gas sensing components, and wherein the MOF materials include HKUST-1, IRMOF-1, MgMOF-74, MOF-177, MOF-801, NU-100, NU-125, UiO-66, and ZIF-8.

5. The gas sensor array system according to claim 2, wherein each of the number of pressure levels is selected from the group consisting of 0.1 bar, 0.5 bar, 1.0 bar, 5.0 bar, and 10.0 bar.

6. The gas sensor array system according to claim 2, wherein the plurality of gas sensing components comprises a first gas sensing component comprising NU-100, a second gas sensing component comprising MOF-177 and a third gas sensing component comprising HKUST-1.

7. The gas sensor array system according to claim 6, wherein number of pressure levels comprises 1 bar.

8. The gas sensor array system according to claim 2, wherein the plurality of gas sensing components comprises a first gas sensing component comprising NU-100, a second gas sensing component comprising IRMOF-1 and a third gas sensing component comprising HKUST-1.

9. The gas sensor array system according to claim 8, wherein number of pressure levels comprises 0.1 bar, 0.5 bar, 1.0 bar, 5.0 bar, and 10.0 bar.

10. A gas sensing method, comprising:
providing a sensor array including a plurality of gas sensing components, wherein each gas sensing component of the plurality of gas sensing components comprises a gas adsorbing material, and wherein the gas adsorbing material is different for each of the plurality of sensing components;
providing a gas sample to the sensor array at number of pressure levels; and
while the gas sample is provided at each pressure level, making a gas parameter measurement on the gas sample using outputs of one or more of the gas sensing components.

11. The method according to claim 10, where each of the gas sensing components comprises a metal oxide framework (MOF) material as the gas adsorbing material, and wherein the MOF is different for each of the plurality of gas sensing components.

12. The method according to claim 11, wherein the plurality of gas sensing components comprises two or more gas sensing components, and wherein the MOF materials include two or more of HKUST-1, IRMOF-1, MgMOF-74, MOF-177, MOF-801, NU-100, NU-125, UiO-66, or ZIF-8.

13. The method according to claim 11, wherein the plurality of gas sensing components comprises nine gas sensing components, and wherein the MOF materials include HKUST-1, IRMOF-1, MgMOF-74, MOF-177, MOF-801, NU-100, NU-125, UiO-66, and ZIF-8.

14. The method according to claim 11, wherein each of the number of pressure levels is selected from the group consisting of 0.1 bar, 0.5 bar, 1.0 bar, 5.0 bar, and 10.0 bar.

15. The method according to claim 11, wherein the plurality of gas sensing components comprises a first gas sensing component comprising NU-100, a second gas sensing component comprising MOF-177 and a third gas sensing component comprising HKUST-1.

16. The method according to claim 15, wherein number of pressure levels comprises 1 bar.

17. The method according to claim 11, wherein the plurality of gas sensing components comprises a first gas sensing component comprising NU-100, a second gas sensing component comprising IRMOF-1 and a third gas sensing component comprising HKUST-1.

18. The method according to claim 17, wherein number of pressure levels comprises 0.1 bar, 0.5 bar, 1.0 bar, 5.0 bar, and 10.0 bar.
